(19) United States
(12) Patent Application Publication
(10) Pub. No.: US 2020/0171267 A1
(43) Pub. Date: Jun. 4, 2020

(54) HEAD WORN DEVICE FOR TREATING NEURODEGENERATIVE DISEASES

(71) Applicants: Matthew D. Millard, Cambridge, MA (US); David A. Gonzales, Lakewood, CO (US)

(72) Inventors: Matthew D. Millard, Cambridge, MA (US); David A. Gonzales, Lakewood, CO (US)

(21) Appl. No.: 16/785,648

(22) Filed: Feb. 10, 2020

Related U.S. Application Data
(63) Continuation-in-part of application No. 15/597,520, filed on May 17, 2017, now abandoned.

(51) Int. Cl. A61M 21/00; A61N 5/06; A61H 23/02; H04R 1/10

(52) U.S. Cl. CPC A61M 21/00; H04R 2460/13; A61H 23/02; H04R 1/1008; H04R 1/1091; H04R 1/1016; A61N 2005/0652; A61M 2021/0055; A61M 2021/0022; A61M 2021/0027; A61N 2005/0629; A61N 2005/0664; A61N 2005/0648; A61M 2205/50; A61M 2209/088; A61M 2230/10; A61M 2230/04; A61M 2210/0693; A61H 2201/10; A61H 2201/1604; A61H 2201/5007; A61H 2201/5012; A61M 2205/3584; A61N 5/0622

(57) ABSTRACT

A head worn device for treating neurogenerative diseases. The head worn device has LEDs mounted to the device. Two printed circuit boards (PCB) are also mounted on the head worn device. Each PCB has a microprocessor and a battery in electrical communication. Each PCB controls a speaker for emitting audio and a vibrating device for emitting a vibration. The LEDs, speaker and vibrating device are in electrical communication with the microprocessor. The microprocessor is programmed to control the LEDs, the speaker and the vibrating device so that each operate at a regulated frequency so that generated light, sound and vibration travel to the user's brain for medical treatment.

The present invention relates devices for treating diseases, and in particular, devices for treating neurodegenerative diseases. The present invention is Continuation-in-Part (CIP) of U.S. patent application Ser. No. 15/597,520, filed May 17, 2017, all of which is herein incorporated by reference.

### BACKGROUND OF THE INVENTION

Neurodegenerative diseases are a very serious problem facing today's society. For example, Alzheimer's dementia is a horrible disease that affects many people. Approximately 5.5 million Americans are currently living with Alzheimer's in 2017. One in ten people age 65 and older currently has Alzheimer's.

Traditionally, Alzheimer's research has focused on the impact of genetics on the disease. In recent years, however, focus has shifted to treating the brain itself. It is known that neurons in the brain interact with each other and will fire at various frequencies. Gamma frequency, defined as the frequency range of 30 Hz to 100 Hz, is important for higher order cognitive function. It has been widely recognized that Alzheimer's patients have diminished neuron activity, especially with respect to the gamma frequency range. Alzheimer's patients also have elevated levels of beta-amyloid peptides. The beta-amyloid peptides are proteins that hinder and block neuron signals, including the gamma oscillation.

At Massachusetts Institute of Technology (MIT) experimentation has been conducted and published that has shown positive results gained after treating mice affected with Alzheimer's. For example, mice suffering with Alzheimer's were exposed to light from Light Emitting Diodes (LEDs) flashing at a gamma frequency of 40 Hz. The mice were placed in a dark area and exposed to a specific frequency light oscillation from LEDs in close proximity. The mice could see the flashing light and it entered their brains through the visual cortex. The visual cortex of the brain is a part of the cerebral cortex that plays an important role in processing visual information.

The treated mice showed remarkable improvement. For example, after an hour of stimulation at 40 Hz, the researchers found a 40 to 50 percent reduction in the levels of beta amyloid proteins in the hippocampus. Additionally, the light exposure stimulated microglia cells. Microglia functions to help remove beta amyloid proteins.

In summary, directly exposing parts of a mouse brain to gamma oscillations supports at least two pathways that aid in the treatment of Alzheimer's. One is to reduce beta amyloid production from neurons. The second is to enhance the removal of beta amyloid by microglia.

More detailed discussions of recent advancements with gamma frequency exposure to Alzheimer infected mice are discussed in the following publications available via the Internet at the following website addresses:
1) http://www.nature.com/nature/journal/v540/n7632/abs/nature20587.html
2) http://www.radiolab.org/story/bringing-gamma-back/
3) http://news.mit.edu/2016/visual-stimulation-treatment-alzheimer-1207
4) http://www.latimes.com/science/sciencenow/la-sci-sn-led-lights-alzheimers-plaques-20161206-story.html

There has not been significant study of the effect of gamma frequency light stimulation on humans. However, the studies on mice lend credence to the hypothesis that a human brain will function similarly to the brain of a mouse and that gamma frequency exposure will reduce beta amyloid production from neurons and enhance the clearance of beta amyloid by microglia. Nevertheless, for the successful treatment of a human, there must be a safe, comfortable, dignified and humane way of providing treatment.

What is needed is an effective way to treat or reduce the effects of neurodegenerative diseases in patients by delivering light and other stimulation to the patient.

### SUMMARY OF THE INVENTION

The present invention provides a head worn device for treating neurogenerative diseases. The head worn device has LEDs mounted to the device. Two printed circuit boards (PCB) are also mounted on the head worn device. Each PCB has a microprocessor and a battery in electrical communication. Each PCB controls a speaker for emitting audio and a vibrating device for emitting a vibration. The LEDs, speaker and vibrating device are in electrical communication with the microprocessor. The microprocessor is programmed to control the LEDs, the speaker and the vibrating device so that each operate at a regulated frequency so that generated light, sound and vibration travel to the user's brain for medical treatment.

### BRIEF DESCRIPTION OF THE DRAWINGS

FIGS. 1 and 3 shows a preferred embodiment of the present invention

FIG. 2 shows a preferred speaker ear piece.

FIG. 4 shows a user wearing a preferred embodiment of the present invention.

FIG. 5 shows another preferred embodiment of the present invention.

FIG. 6 shows another preferred embodiment of the present invention.

FIG. 7 shows another preferred embodiment of the present invention.

FIG. 8 shows another preferred embodiment of the present invention.

FIG. 9 shows another preferred embodiment of the present invention.

FIGS. 10-15 show other preferred embodiments of the present invention.

### DETAILED DESCRIPTION OF THE PREFERRED EMBODIMENTS

In preferred embodiments of the present invention LEDs are mounted on eyeglass frames so that the LEDs are in close proximity to the user. The LEDs are programmed to flash at a gamma frequency of 30-100 Hz. For example, in one preferred embodiment the LEDs flash at 40 Hz. The user is therefore able to easily be exposed to the flashing light. The light enters the user's brain through the eyes and travels to the visual cortex. The user's brain is then exposed in a manner similar to that discussed above allowing a treatment modality for a person suffering from Alzheimer's disease by reducing beta amyloid production from neurons and enhancing the clearance of beta amyloid by microglia.

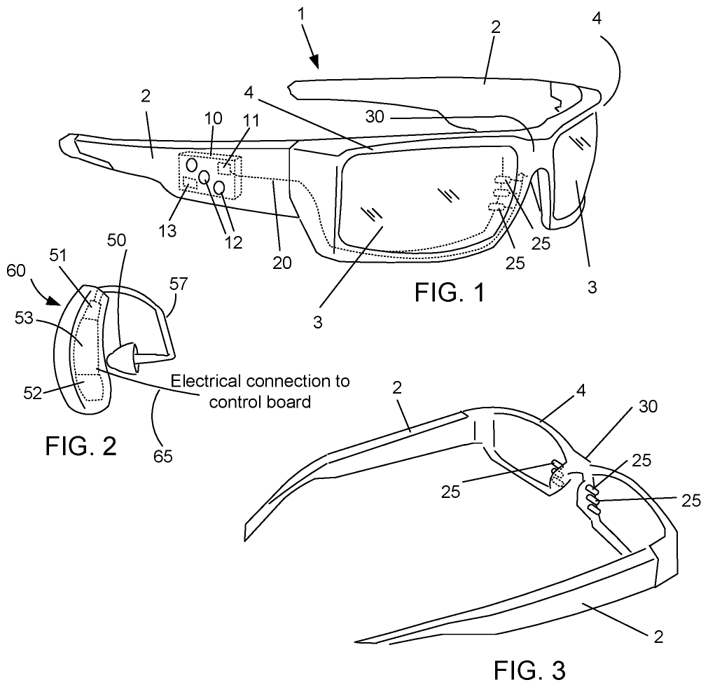

FIG. 1 shows a first preferred embodiment of the present invention. Glasses 1 include temples 2, lenses 3, and lens frame 4. Printed circuit board 10 is mounted into temple 2 as shown. PCB 10 includes microprocessor 11, control switches 12 and battery 13, each of which is in electrical communication with one another. Electric wire 20 extends from PCB 10 and runs through temples 2 and frames 4 and connects microprocessor 11 to LEDs 25, as shown. It should be noted that in another preferred embodiment control switches 12 can be replaced with a remote control device.

Microprocessor 11 is programmed to control the flashing rate of LEDs 25 so that they oscillate at 40 Hz or in the gamma frequency range. In a preferred embodiment, the user can turn LEDs 25 on or off by using control switches 12. Also in a preferred embodiment the user can vary the oscillation rate of LEDs 25 with switches 12. In a preferred embodiment LEDs 25 will always oscillate within the gamma frequency range, at a value between 30 Hz and 100 Hz.

As shown in FIGS. 1 and 3, LEDs 25 are mounted on frame 4 near nose support 30. However, it should be noted that LEDs 25 may be mounted anywhere on frame 4 behind lenses 3 so that flashing light from LEDs 25 is able to enter the eyes of the user and travel to the visual cortex for Alzheimer's treatment.

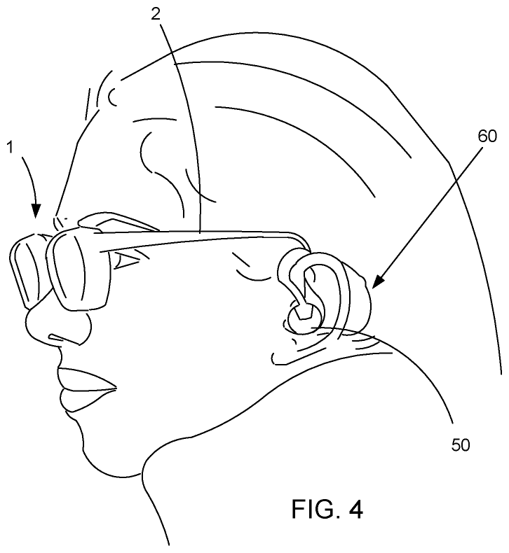

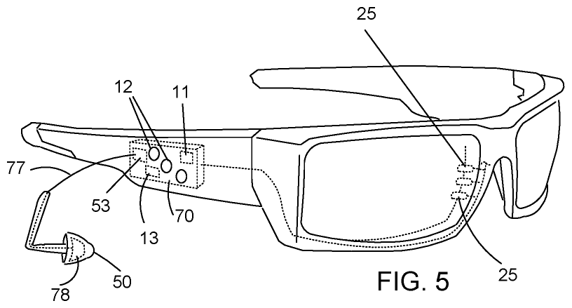

In another preferred embodiment shown in FIG. 2, ear mold 50 is inserted into a user's ear. Speaker earpiece 60 includes battery 52, amplifier 53 and speaker 51. Amplifier 53 is in electrical communication with PCB 10 and microprocessor 11 via wire 65. Ear mold 50 receives audio from speaker 51 via tubing 57. In a preferred embodiment, ear mold 50 transmits oscillating audio to the user that pulsates at the gamma frequency, preferably 40 Hz, or other frequency. The audio is heard by the user through the ear, traveling to the user's brain. In a preferred embodiment, the audio is preferably in sync with the flashing light at 40 Hz. FIG. 4 shows a side view of a user donning glasses 1 also using speaker earpiece 60 with ear mold 50 inserted into her ear.

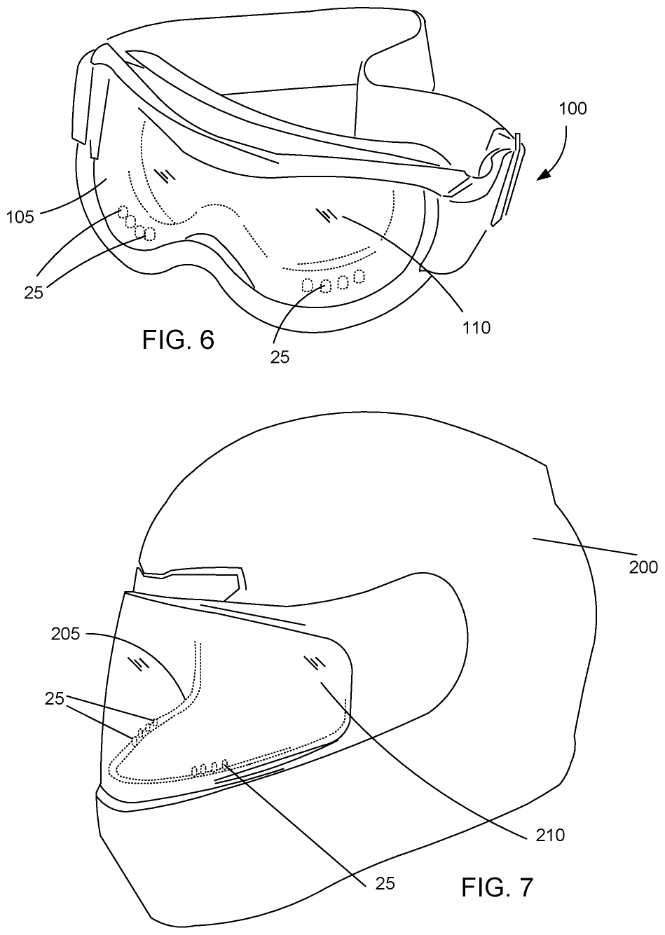

FIG. 5 shows another preferred embodiment of the present invention. PCB 70 includes microprocessor 11, control switches 12 and battery 13, each of which is in electrical communication with one another. PCB 70 also includes amplifier 53. Speaker wire 77 connects amplifier 53 to speaker 78 in ear mold 50. In a preferred embodiment, ear mold 50 transmits oscillating audio to the user that pulsates, preferably in the gamma frequency, or more specifically at 40 Hz. The audio is heard by the user through his ear and travels to the user's brain. The audio is preferably in sync with the flashing light from LEDs 25 at 40 Hz.

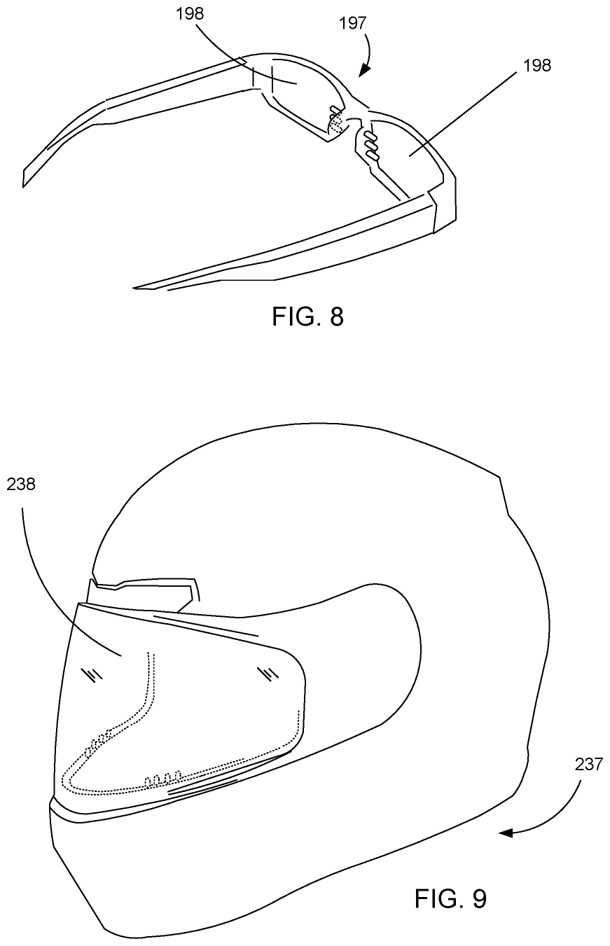

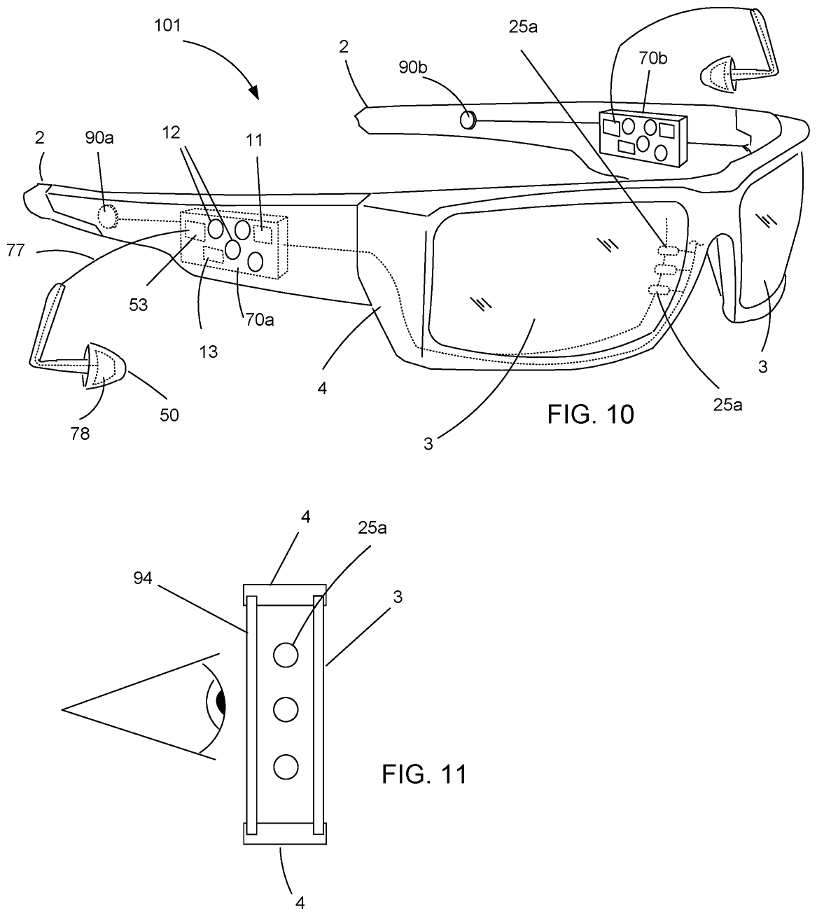

The above preferred embodiments showed LEDs 25 mounted on eye glasses. Eye glasses are comfortable to wear and can be worn with dignity and ease. It also should be noted that there are other types of head worn devices that may also be utilized with similar effectiveness. For example, FIG. 6 shows LEDs 25 mounted on goggle frame 105 of goggles 100 behind lens 110. As with eye glasses 1, flashing light from LEDs 25 is able to enter the eyes of the user and travel to the visual cortex for Alzheimer's treatment. Also, FIG. 7 shows LEDs 25 mounted on helmet frame 205 of helmet 200 behind lens 210. As with eye glasses 1, flashing light from LEDs 25 is able to enter the eyes of the user and travel to the visual cortex for Alzheimer's treatment.

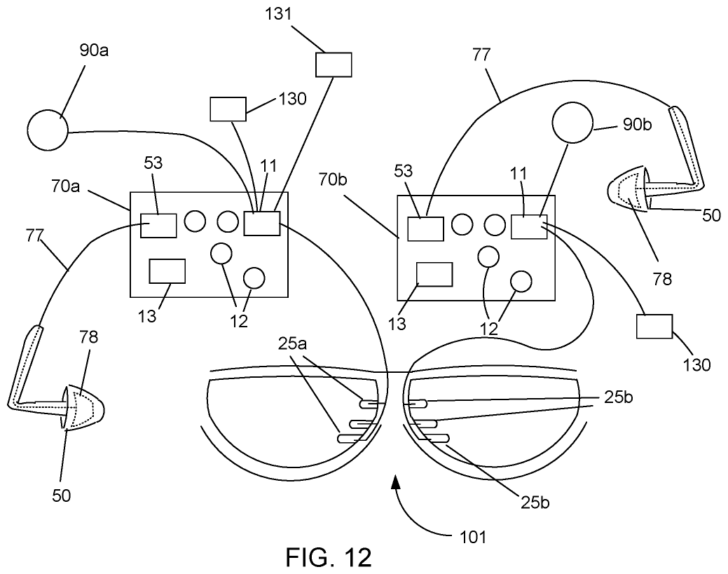

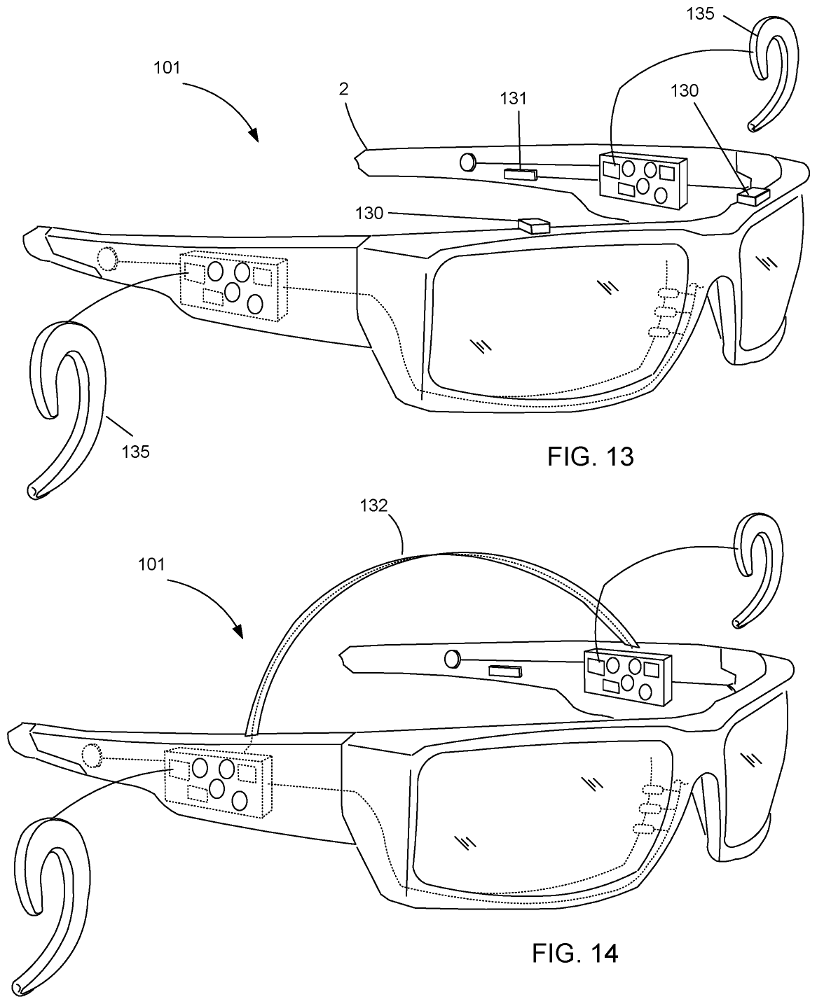

Above it was explained that lenses 3 allow a user to see through eye glasses 1 while being treated. In another preferred embodiment lenses are omitted and instead the user's eyes are covered by an opaque covering. For example, FIG. 8 shows head worn device 197 having opaque lenses 198. Also, FIG. 9 shows head worn device 237 having opaque lens 238. It would also be possible to utilize a head worn device in the shape of a box as well, having no lenses and with the LEDs attached directly to the head worn device.

### Other Preferred Embodiments

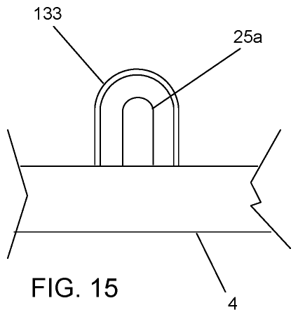

FIGS. 10-12 show another preferred embodiment of the present invention. Glasses 101 include vibrating mini motor discs 90a and 90b and programming so that discs 90a and 90b may be used to vibrate at gamma frequency for the treatment of Alzheimer's.

Glasses 101 include PCB 70a and PCB 70b. PCBs 70a and 70b are similar to PCB 70 described above. PCB 70a and 70b each include microprocessor 11, control switches 12 and battery 13, each of which is in electrical communication with one another. PCBs 70a and 70b also each include an amplifier 53. Speaker wire 77 connects amplifier 53 to speaker 78 in ear mold 50. In a preferred embodiment, ear mold 50 transmits oscillating audio to the user that pulsates, preferably in the gamma frequency, or more specifically at 40 Hz. The audio is heard by the user through his ear and travels to the user's brain. In one preferred embodiment the audio is preferably in sync with the flashing light from LEDs 25a and 25b at 40 Hz.

As stated above, glasses 101 further includes two vibrating mini motor discs 90a and 90b, each one connected to a temple 2. Disc 90a is electrically connected to PCB 70a and disc 90b is electrically connected to PCB 70b. Microprocessors 11 are programmed to send electrical control signals to vibrating mini motor discs 90a and 90b. In one preferred embodiment discs 90a and 90b are programmed to vibrate at gamma frequency. For example, in one preferred embodiment discs 90a and 90b vibrate in sync with flashing light from LEDs 25a and 25b at 40 Hz. The vibration is felt by the wearer of glasses 101 and may be used for Alzheimer's treatment.

As shown in FIGS. 10 and 12, glasses 101 include two printed circuit boards (PCB 70a and 70b), each PCB allows for independent control of one of the sides of glasses 101. Accordingly, two independent circuits are able to control multiple entry ways to the brain center of the user. For example, by manipulating control switches 12 the user can vary LED light frequency and LED light wavelength from LEDS 25a and 25b. Also, by using control switches 12, the user can vary audio signals emitted from speakers 78 independently of one another. Additionally, control switches 12 allow the user to control the vibration frequency emitted from discs 90a and 90b.

It should be further noted that glasses 101 includes two independently controlled light frequency sources (LEDs 25a and LEDs 25b). Hence, microprocessors 11 can be programmed to control LEDs 25a and LEDs 25b to emit varying frequencies for the user. Gamma frequency (30 Hz to 100 Hz) encompasses a broad range. Multiple frequencies may easily be programmed within the gamma frequency range. For example, PCB 70a may be programmed to control LEDs 25a to emit light at 45 Hz, while simultaneously PCB 70b is programmed to control LEDs 25b to emit light at 80 Hz.

Also, in a preferred embodiment glasses 101 include interior lens 94, as shown in FIG. 11. In a preferred embodiment, lens 94 is tinted and functions to diminish the brightness of light from LEDs 25a and 25b reaching the user's eye. In a preferred embodiment, lens 94 is tinted plastic and is removeable and interchangeable. In another preferred embodiment lens 94 is tinted glass. The user may swap out different lenses 94 as appropriate to vary the level of brightness reaching the eye.

### Brain Monitor and Pulse Monitor

FIG. 13 shows brain monitor sensors 130. Brain monitor sensors 130 are used to monitor the brain waves of the user and as a treatment and research device. Pulse sensor 131 is used to monitor the pulse of the user. Data from sensors 130 and 131 may be collected and analyzed and used to generate information of patients with neurodegenerative diseases in the absence of a hospital and medical office setting. This is extremely beneficial in the search for causes of neurodegenerative diseases and potential solutions. FIG. 14 shows alternative brain monitor sensor 132.

### Controlling Lux with a Diffuser

FIG. 15 shows LED 25a connected to frame 4 and covered by diffuser 133. Lux is the standard SI unit of measurement for illuminance. Diffuser 133 allows a general low voltage LED 25a to be dimmed, thereby lowering the illuminance to a desirable level. LED's operates on a duty cycle, and although circuitry can be used to adjust the duty cycle and therefore lower total Lux, it has serious limitations. Diffuser 133 enables complete control over the Lux, in that it will dim LED 25a and lower the illuminance well below what the duty cycle can achieve. This allows for low levels of light, much below what can be achieved without diffuser 133.

Although the above-preferred embodiments have been described with specificity, persons skilled in this art will recognize that many changes to the specific embodiments disclosed above could be made without departing from the spirit of the invention. For example, even though the above preferred embodiments discussed LEDs flashing at 40 Hz, it would be possible to adjust the frequency of the flashing to a different value, preferably in the gamma frequency range of 30 to 100 Hz. In another preferred embodiment the LEDS are programmed to flash at a frequency outside the gamma frequency range. It was described above how the utilization of tinted lens 94 allows the user to control the brightness of LEDs 25a and 25b. In another preferred embodiment, the brightness of light emitted from LEDs 25a and 25b is controlled electronically via control switches 12. Also, although above it was described how the present invention is primarily used to treat Alzheimer's, it may also be utilized to treat other major issues that arise from the brain center of the user. It should be understood that frequency control can be achieved other ways than described above. For example, in one preferred embodiment a 555 timer circuit is utilized. The 555 timer circuit allows for controlling specific wavelength, duty, pulse rate, and limited amplitude. Also, although it was described above how physical vibration is utilized for gamma frequencies, it should be understood that vibrating discs 90a and 90b may be used to generate frequencies above and below gamma frequencies. Also, even though the above preferred embodiments discussed the utilization of ear mold 50, it should be understood that other types of audio sound devices may be used, such as bone conducting headphones 135 (FIG. 13). Also, it should be understood that the head worn device 101 may be used as a research tool. It may be used to monitor patients and collect data. By utilization of the present invention there is no need for hospital visits, a research lab or doctor office visits. Software can be utilized to send data to a centralized cloud system, thereby allowing for proper data analysis. Additionally, the patient can adjust on their own or load preset programs from others or from the main system provider. Therefore, the attached claims and their legal equivalents should determine the scope of the invention.

What is claimed is:

1. A head worn device for treating neurodegenerative diseases, comprising:
A. a plurality of LEDs mounted to said head worn device,
B. at least one PCB mounted on said head worn device, said at least one PCB comprising:
i. a microprocessor,
ii. a battery, wherein said microprocessor said battery are each in electrical communication, wherein said plurality of LEDs are in electrical communication with said microprocessor,
C. a speaker in electrical communication with said microprocessor, said speaker generating audio frequencies,
D. a sound transmission device for placement next to a user's ear, said sound transmission device for receiving said audio frequencies and for transmitting to the user said audio frequencies,
E. a vibrating device mounted onto said head worn device and in electrical communication with said microprocessor,
wherein said plurality of LEDs are programmed to flash at a regulated frequency, wherein said speaker generates audio frequencies at a regulated frequency, and wherein said vibrating device vibrates at a regulated frequency, so that the light from said LEDs, the sound from said speaker, and the vibration from said vibration device travels to the brain of the user for therapeutic treatment.

2. The head worn device as in claim 1, wherein said plurality of LEDs flash at gamma frequency, wherein said speaker generates audio signals at gamma frequency and said vibration device vibrates at gamma frequency for the treatment of Alzheimer's.

3. The head worn device as in claim 1, further comprising:
A. an interior tinted lens between said plurality of LEDs and the user's eye to control LED brightness,
B. an exterior lens to permit viewing, and
C. a frame for supporting said interior lens and said exterior lens, wherein said plurality of LEDs is mounted to said frame.

4. The head worn device as in claim 1, wherein said interior lens is removably attached.

5. The head worn device as in claim 1, further comprising at least one control switch in electrical communication with said microprocessor for controlling the flashing of said plurality of LEDs, electrical signals sent to said speaker, electrical signals sent to said vibrating device.

6. The head worn device as in claim 1 wherein said vibrating device is a vibrating mini motor disc.

7. The head worn device as in claim 1, further comprising a remote-control device in electrical communication with said microprocessor for controlling the flashing of said plurality of LEDs, said speaker and said vibrating device.

8. The head worn device as in claim 2, wherein said gamma frequency is 40 Hz.

9. The head worn device as in claim 1, wherein said head worn device is eye glasses comprising temples connected to said frame, wherein said PCB is mounted onto said temples.

10. The head worn device as in claim 1, wherein said head worn device is goggles.

11. The head worn device as in claim 1, wherein said head worn device is a helmet.

12. The head worn device as in claim 1, wherein said at least one PCB is two PCBs mounted onto said frame, each of said two PCBs for independent control of said LEDs, said speaker and said vibrating device on one side of said head worn device.

13. The head worn device as in claim 12, wherein said plurality of LEDs comprises at least two separate independently controlled light frequency sources, wherein each said independently controlled light frequency source is capable of emitting a light frequency having a value distinct from the other independently controlled light frequency source so that multiple light frequencies may be simultaneously emitted from said plurality of LEDs.

14. The head worn device as in claim 1, further comprising brain monitor sensors for monitoring brain waves.

15. The head worn device as in claim 1, further comprising a pulse monitor for monitoring the pulse of the user.

16. The head worn device as in claim 1, wherein said sound transmission device is an ear mold.

17. The head worn device as in claim 1, wherein said sound transmission device is a bone conducting headphone.

18. The head worn device as in claim 1, further comprising a diffuser covering said plurality of LEDs.

19. The head worn device as in claim 1, wherein software is utilized to send data to a centralized cloud system for data analysis.

20. A head worn device for treating neurodegenerative diseases, comprising:
A. a plurality of LEDs mounted to said head worn device,
B. at least one PCB mounted on said head worn device, said at least one PCB comprising:
i. a microprocessor,
ii. a battery, wherein said microprocessor said battery are each in electrical communication, wherein said plurality of LEDs are in electrical communication with said microprocessor,
C. a speaker in electrical communication with said microprocessor, said speaker generating audio frequencies,
D. a sound transmission device for placement next to a user's ear, said sound transmission device for receiving said audio frequencies and for transmitting to the user said audio frequencies,
E. a vibrating device mounted onto said head worn device and in electrical communication with said microprocessor,
F. a brain monitor sensor for monitoring brain waves, said brain monitor in electrical communication with said microprocessor,
G. a pulse monitor for monitoring the pulse of the user, said pulse monitor in electrical communication with said microprocessor, and
H. a diffuser covering said plurality of LEDs,
wherein said plurality of LEDs are programmed to flash at a regulated frequency, wherein said speaker generates audio frequencies at a regulated frequency, and wherein said vibrating device vibrates at a regulated frequency, so that the light from said LEDs, the sound from said speaker, and the vibration from said vibration device travels to the brain of the user for medical treatment.
